(19) United States
(12) Patent Application Publication
(10) Pub. No.: US 2005/0020997 A1
(43) Pub. Date: Jan. 27, 2005

(54) Method and apparatus for anesthetizing a uterus

(76) Inventor: Thierry G. Vancaillie, Castlecrag (AU)

Correspondence Address: Eppa Hite, 2318 Louis Road, Palo Alto, CA, 94303, US

(21) Appl. No.: 10/625,816

(22) Filed: Jul. 22, 2003

(51) Int. Cl. A61M 31/00

(52) U.S. Cl. 604/506; 604/279

(57) ABSTRACT

A method of applying topical anesthetic to the inner surfaces of the uterus and cervix and a delivery device for practicing the method. The delivery device has a hollow discharge tube which is preferably curved towards a distal tip with at least one aperture. The tube is inserted through the vagina and the cervical canal until the resistance of the fundus is perceived. The delivery device is then operated to discharge anesthetic material through the aperture(s) in the region of the fundus, the tubal ostia and the cornua of the uterus such that the material displaces intra-uterine debris to facilitate reaching the mucosal surfaces of the uterus.

### BACKGROUND OF THE INVENTION

1. Field of the Invention

The present invention relates generally to anesthesiology, and more particularly to a method and apparatus for applying topical anesthetic to a woman's uterus.

2. Discussion of Prior Art

Hysteroscopy enables gynecologists to perform minimally invasive procedures to address common problems such as polyps and fibroids that occur within the uterus. Additionally, hysteroscopes in their many forms facilitate medical procedures that until recently were quite complicated. New medical devices that require the use of specialized catheters, with or without a hysteroscope, have been introduced in recent years that make it easier to perform some procedures such as endometrial ablations (e.g., hot water balloon) and female sterilizations (e.g., Essure). Many of these new procedures should be done in an office setting or on an out-patient basis using some form of local anesthesia, allowing the patient after a short recovery period to return home. Other new products are in development and will soon be brought to market.

In preparation for performing intra-uterine examinations, manipulations and/or surgical procedures, gynecologists sometimes have elected not to use any anesthetic in view of the short duration of such events, or conventionally have elected to inject a local anesthetic into tissue around and near the cervix. This results in what is termed a paracervical block (PCB). In injecting an anesthetic into tissue, PCB increases the risk that the patient may be exposed to a certain degree of morbidity, due to the relatively high probability that some of the injected drug will find its way into the vascular system.

Morbidity associated with local anesthesia is due to the mode of administration or to the drug itself. The toxicity of the drug, although minimal, encompasses anaphyllaxis and extremely rare conditions such as methemoglobinemia, both potentially fatal. Drug toxicity exists no matter how anesthetic is applied, whether by topical application or injection. Morbidity due to the mode of administration is related to the speed of absorption of the drug into the vascular system and the ensuing effect anesthetic drugs have on the cardiovascular, central nervous and respiratory systems. The effects vary from a metallic taste in the mouth, through palpitations, to cardiac arrest, and death. The likelihood of intravascular absorption is significantly lower with topical application (i.e. placed upon the surface) as compared to infiltration of the compounds directly into the tissues with devices such as needles.

H. M. Hasson in an article entitled “Topical Uterine Anesthesia: A Preliminary Report” published in the International Journal of Gynaecology & Obstetrics Vol. 15 pp 238-240 (1977) taught the instillation of lidocaine in aqueous solution, through a cannula into the cervical canal, as a topical anesthetic method for reducing pain associated with intra-uterine manipulations during gynecologic procedures. However, an anesthetic drug in aqueous solution is limited in its effectiveness by the fact that the uterus is normally occupied with mucus, blood and tissue debris which block the liquid anesthetic from reaching the inner surfaces of all the parts of the uterus where it is supposed to act. Further, even if the cervix is sealed by a solid acorn around the cannula to impede backflow of the aqueous solution, the thin liquid anesthetic will not be capable of displacing the more viscous mucus and other tissular substances covering the surfaces of the cervical and uterine cavities. Direct contact between the anesthetic agent and the tissue to be anesthetized is a prerequisite for the action of topical anesthetic drugs. Anesthetic drugs in aqueous solution instilled into the cervical cavity have therefore minimal capability to succeed in their objective, due to their unpredictable effectiveness in reaching the tissues to be anesthetized.

The PCB method commonly used for gynecological operations injects anesthetic drugs in aqueous solution directly into the tissues around and near the cervix to control pain while a catheter, an instrument or a hysteroscope is inserted through the cervical canal into the uterus and some intervention is carried out. With PCB (local infiltration) the incidence of morbidity due to the mode of administration varies depending upon a variety of factors, such as age, time in menstrual cycle, depth of insertion of the needle, and the presence of inflammation or other cause of vascular distention at the site of application.

However the PCB method affects only the nerves whose course originates along the uterine vessels and passes down along the cervix and up along the lower portion of the uterus. It does not affect the nerves whose course originates along the ovarian vessels toward the corpus of the uterus, the fundus and the comu containing the tubal ostium. The exact distribution of innervation is variable from one patient to the other, which explains the variability of the PCB in relieving pain for various intra-uterine interventions. However, the tubal ostium is more or less consistently dependant on innervation coming down the ovarian pedicle. Local anesthesia using only a PCB results in patients experiencing considerable discomfort when procedures are performed in the cornual areas of the uterus, for instance for the purpose of performing a sterilization. Thus the critical concern when considering whether to use PCB is that it should address the area of intended surgery. PCB is sometimes an inappropriate mode of local anesthesia. For example in a hysteroscopic sterilization PCB will numb the cervix and the lower portion of the uterine corpus but inconsistently the mid and upper areas of the corpus and the fundus and not the cornua of the uterus. PCB only addresses the pain induced by passage of the hysteroscope or catheter through the cervix, not the pain caused by the hysteroscopic procedure itself. Moreover, if a 4 mm or smaller hysteroscope is used, PCB is probably unwarranted for reducing pain caused by entry through the cervix and is certainly ineffective for reducing pain caused by performing the actual procedure at the level of the ostium.

There remains, therefore, a need for an improved method of anesthetizing a uterus.

### SUMMARY OF THE INVENTION

It is therefore an objective of the present invention to provide a method for topical anesthesia inside the uterus to provide anesthetic cover for some common gynecological procedures and for emerging procedures that involve those parts of the uterus which are not well covered by traditional approaches.

It is another objective to offer physicians an effective and easy-to-use means to achieve anesthetic cover as an alternative to the established paracervical block (PCB), which is perceived by many gynecologists as either too dangerous or excessive for minor intra-uterine manipulations, despite the fact that these manipulations induce short-lived pain.

A preferred method according to the present invention uses a reservoir which is pre-filled with anesthetic material and is either attached, or connectable, to a catheter which can be inserted through the cervix into the uterus to reach the fundus and the left and right ostium. Once the reservoir-catheter assembly has been placed in position, the operator can deliver the anesthetic material at the site of intended action. The action of delivering the anesthetic material leads to displacement of the mucus and tissular debris which covers the inner surfaces of the uterus. This debris is pushed out of the way and allowed to exit through a partially open cervical canal. The anesthetic material now covers the inner surfaces of the uterine cavity. The viscosity of the material, being higher than that of water, allows the material to remain in the hollow of the uterine cavity for several minutes. During that time the anesthetic drug contained in the material diffuses into the inner uterine tissue layers, reaching the nerve endings which are the intended target of anesthetization. After a latency period of several minutes (which is a function of the type of drug incorporated in the material) the tissues are sufficiently numbed to proceed with minor surgical manipulations and procedures.

Among the advantages of the invention are that the procedure is simple to perform; topical anesthetic drugs carry a lower risk for complications, which will reduce the reluctance of many gynecologists to offer their patients effective pain relief; the anesthetic drug is delivered at the site of intended surgery in cases of specific procedures such as hysteroscopic tubal occlusion procedures; and the procedure can be combined with the existing PCB to increase the latter's effectiveness.

These and other advantages of the present invention will become more apparent to those skilled in the art upon reading the following description of the preferred embodiment.

### BRIEF DESCRIPTION OF THE DRAWING

FIG. 1 is a side view of a one-piece syringe or container/applicator with a straight cannula;

FIG. 2 is a side view of a one-piece syringe or container/applicator with a curved cannula;

FIG. 3 is a side view of a two-piece syringe including a plunger and a one-piece cylinder and straight cannula;

FIG. 4 is a side view of a two-piece syringe including a plunger and a one-piece cylinder and curved cannula;

FIG. 5 is a side view of a straight cannula and a curved cannula, either of which may be attached to a conventional two-piece syringe including a cylinder and a plunger;

FIG. 6 shows a side view of a curved cannula and a cross-section of a two-piece syringe including a cartridge pre-filled with medication and a cylinder with a reverse needle which punctures a cap on the cartridge when the cylinder slides over the cartridge; and

FIG. 7 is a cut-away view of a uterus and a syringe as in FIG. 4 being used to apply topical anesthetic to the fundus of a uterus.

### DETAILED DESCRIPTION OF THE INVENTION

The disclosure of U.S. Pat. No. 6,423,038 is hereby incorporated by reference.

The present invention provides a new method for anesthetizing a woman's uterus. The method may be used prior to commonly performed medical examinations and surgical procedures such as:
Endometrial biopsies;
Office hysteroscopies;
Endometrial ablations (second generation, such as with hot water balloons);
Hysteroscopic sterilizations;
Dilatation and curettage (D&C, diagnostic as well as therapeutic);
Endometrial biopsies, screening for endometrial cancer; and
Insertion or removal of Intra-Uterine Contraceptive Devices (IUCDs).

The invention also provides an apparatus comprising a syringe or container/applicator which is conveniently preloaded with a material containing an anesthetic drug such as lidocaine hydrochloride or another suitable anesthetic, in the form of a gel or otherwise having a viscosity greater than that of water, and a catheter or cannula tube.

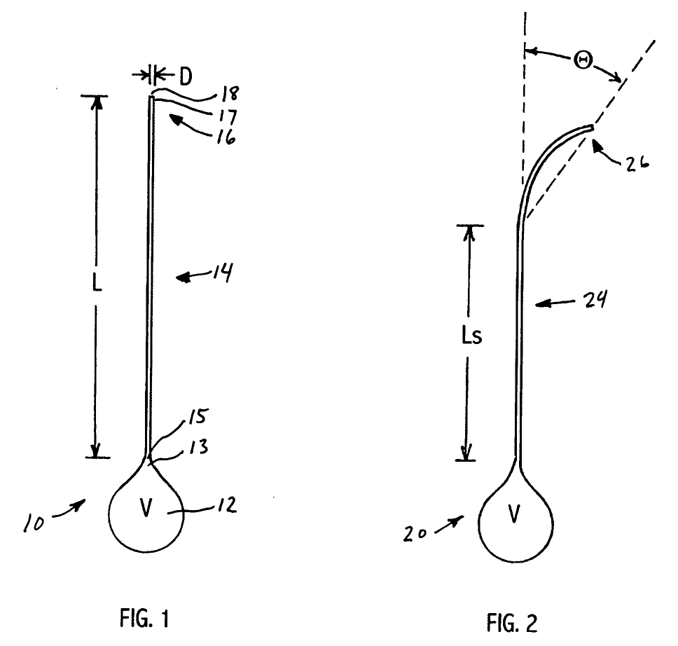

FIG. 1 is a side view of a one-piece container/applicator 10 with a deformable cannula tube for applying topical anesthetic to the fundus of a uterus. The container/applicator 10 includes a reservoir 12 capable of holding a volume V, preferably of the order of 5 cc, of relatively viscous anesthetic. The reservoir 12 has an outlet 13 for discharging the anesthetic. The container/applicator further includes a catheter or hollow tube 14 having a proximal end 15 for receiving the anesthetic from the outlet and a distal end 16 with a blunt tip 17 forming at least one aperture 18, in the end and/or in the wall of the tube, for discharging anesthetic from the tube. The tube 14 length L, approximately 22 to 30 cm, is sufficient to extend through a vagina and a cervical canal into a uterus (not shown) to its fundus, and the tube 14 diameter D, approximately 3 to 4 mm, is sufficiently small to allow tissular debris and secretions as well as excess discharged anesthetic to flow from the uterus along the outside of the tube 14 through and out the cervical canal.

FIG. 2 is a side view of a one-piece container/applicator 20 wherein the tube 24 has a straight length Ls and, from midway between the ends towards the distal end 26, is curved to an angle Θ. This angle is in the order of 30 degrees.

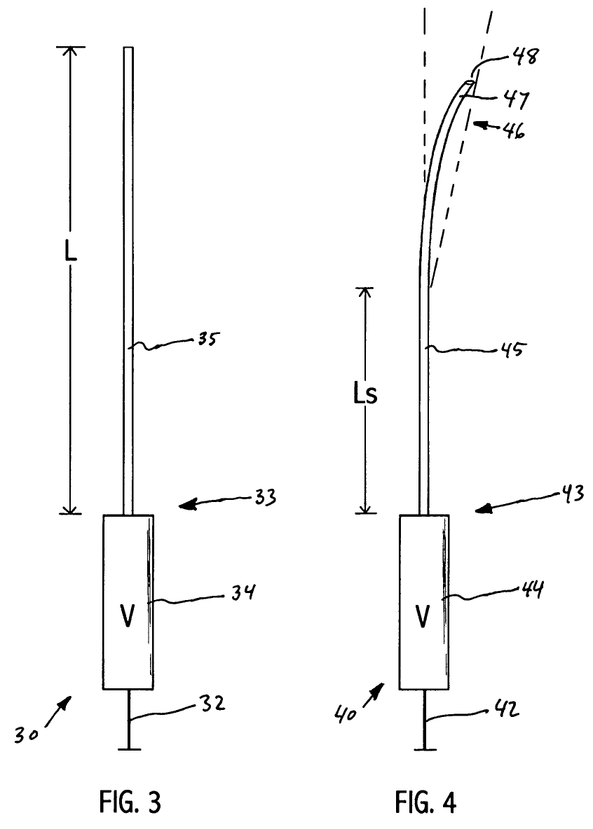

FIG. 3 is a side view of a container/applicator in the form of a two-piece syringe 30 including a plunger 32 and a one-piece cylinder/tube 33 including cylinder 34 and straight cannula 35.

FIG. 4 is a side view of a container/applicator in the form of a two-piece syringe 40 including a plunger 42 and a one-piece cylinder/tube 43 including cylinder 44 and curved cannula 45 whose distal end 46 has a tip 47 with at least one aperture 48.

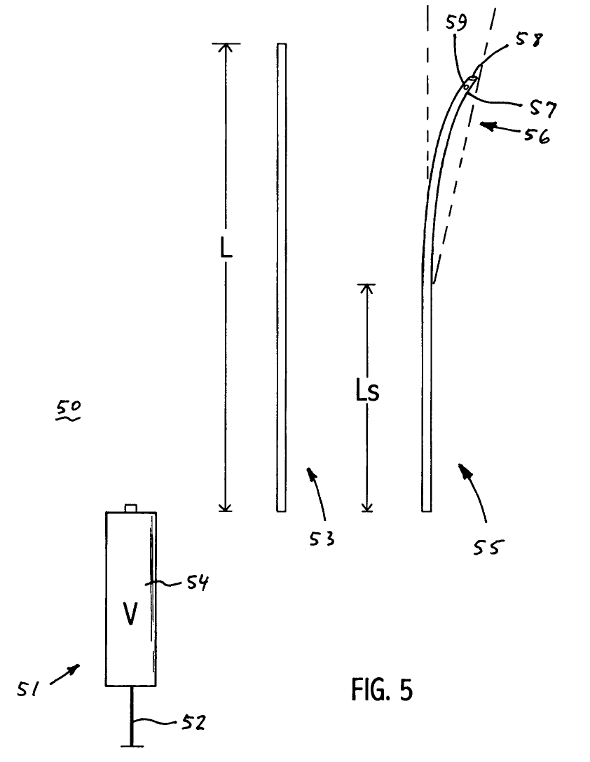

FIG. 5 illustrates a container/applicator 50 in the form of a two-piece syringe 51 including a plunger 52 and a cylinder 54 which is attachable to a cannula. The syringe 51 may be attached to either a straight cannula 53 or a curved cannula 55. Curved cannula 55 at its distal end 56 has a tip 57 with one aperture 58 across the axis of the tube and another aperture 59 along the axis of the tube

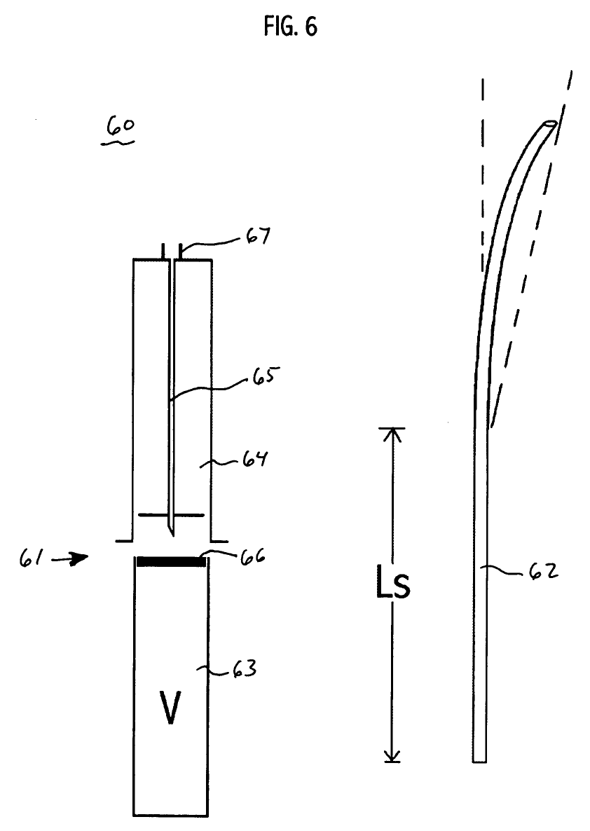

FIG. 6 illustrates a container/applicator 60 in the form of a two-piece syringe 61 and a curved cannula 62. The two-piece syringe includes a pre-filled cartridge 63 and a cylinder 64 with a reverse needle 65. When the cylinder 64 is slid over the cartridge 63 the needle 65 punctures a cartridge cap 66 and drives the cap into the cartridge 63, forcing the medication into the reverse needle and out a syringe nozzle 67 into the cannula 62. Two-piece syringes similar to this and pre-filled with 2% lidocaine gel are sold under the name Uro-Jet® by International Medication Systems, or Amphastar, of Rancho Cucamonga, Calif. The Uro-Jet® is used with an attachment for instillation in the bladder.

Other self-contained liquid dispensing devices are described in U.S. Pat. No. 4,875,602 by Chickering et al.

In Operation

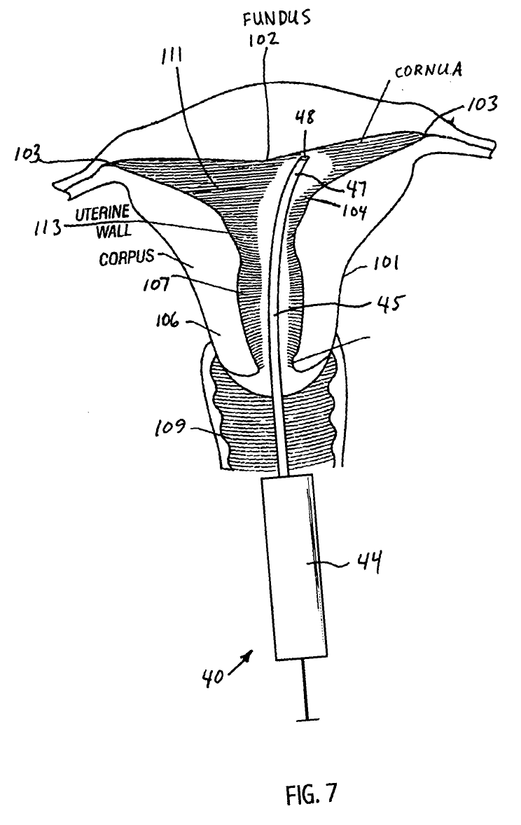

A syringe according to one of the FIGS. 1 through 6, or another container/applicator configuration satisfying the requirements of the invention, may be used to practice the method of the invention. For example, FIG. 7 is a cut-away view of a uterus 101 and a syringe 40 as shown in FIG. 4.

To topically anesthetize a uterus or part thereof, according to the method of the invention, a few minutes before the procedure is to begin, the examining physician, surgeon or nurse inserts a cannula 45 through the vagina 109 and the cervical canal 107 to position the cannula tip 47 proximate the fundus 102 of the uterus 101 or proximate either one of the tubal ostia 103. Then a vehicle (e.g., a gel) with viscosity greater than water containing an anesthetic drug, such as lidocaine, marcaine or another such compound, is delivered from the reservoir 44 through the cannula 45 and discharged out the aperture 48 onto and near the fundus 102 of the uterus 101. The tip 47 may also be positioned to direct the discharge of anesthetic towards either or both of the tubal ostia 103. During the maneuver of discharging the vehicle containing the anesthetic, the cannula 45 is moved from side to side while being withdrawn, to ensure even distribution of the material within the uterine 111 and cervical 107 cavities.

While the vehicle containing the anesthetic is being discharged through one or more apertures 48 in the tip 47, the vehicle disperses evenly within the uterine cavity 111 and can backflow out of the uterus 101 along the outside of the cannula 45 through the cervical canal 107. This controlled flow of material will flush naturally occurring debris from the mucosal surface 113 of the uterus, out of the uterine cavity 111 and cervix 106, and facilitate the injected material reaching the inner surfaces of the uterine corpus 104, fundus 102, cornua as well as the inner surfaces of the tubal ostia 103 and cervical canal 107. Such backflow along the cannula 45 may indicate the sufficiency of use of the material. The anesthetic drug contained within the vehicle then diffuses into the tissues and reaches the nerve endings contained within the inner layers of the uterine organ 101 at all locations where the material was deposited. The anesthetic drug then effects its action onto the nerve endings at variable latencies, depending on the drug being used. The onset of action for inducing anesthesia typically is delayed a few minutes after insertion of the catheter 45 into the uterine cavity 111. Thereafter, minor intra-uterine manipulations and interventions such as hysteroscopy, endometrial biopsy, insertion of an intra-uterine device or performance of a trans-cervical sterilization become possible with an acceptable level of patient discomfort, if any.

When a hysteroscope, catheter, or other instrument greater than 4 mm in diameter is to be introduced through the cervix for the performance of a particular procedure, paracervical block (PCB) may be indicated in addition to the use of the inventive method. Combination of the established PCB with intra-uterine topical anesthesia will most likely address the analgesic needs of the patient undergoing procedures such as with the second generation endometrial ablation devices. These procedures need the paralysis of nerve endings originating from both the uterine and ovarian pedicles. This can effectively be achieved by combining the established PCB of the uterine fibers with in utero instillation of an anesthetic material to address the nerve endings reaching the uterus via the ovarian pedicle.

While the present invention is described in terms of a preferred embodiment, it will be appreciated by those skilled in the art that this embodiment may be modified without departing from the essence of the invention. It is therefore intended that the following claims be interpreted as covering any modifications falling within the true spirit and scope of the invention.

1. A method of anesthetizing a uterus, comprising the steps of:
inserting a cannula having a tip through a cervical canal;
positioning the cannula so that an aperture in the tip is proximate the fundus of the uterus; and
delivering anesthetic through the cannula and out the aperture to the fundus.

2. The method of claim 1 wherein positioning directs the tip towards a tubal ostia.

3. The method of claim 1 comprising the step of, after delivering the anesthetic out the aperture, permitting the anesthetic to flow out of the uterus alongside the cannula through the cervical canal.

4. The method of claim 1 wherein the anesthetic has a viscosity greater than that of water.

5. The method of claim 1 wherein the anesthetic is in the form of a gel.

6. The method of claim 1 wherein the anesthetic comprises a compound of the lidocaine family.

7. A container/applicator apparatus for applying topical anesthetic to the middle to upper corpus regions, the fundus, and the tubal ostia of a uterus, comprising:
a reservoir for containing anesthetic and having a reservoir outlet for releasing the anesthetic; and
a hollow tube having
a proximal end for receiving the anesthetic released from the outlet,
a distal end with a tip,
a length between the proximal end and the tip sufficient to extend through a vagina and a cervical canal into a uterus to its fundus, and
at least one aperture in the tip for discharging the anesthetic from the tube.

8. The apparatus of claim 7 wherein the tube has an axial cross-section sufficiently small to allow fluid to flow from the uterus alongside the tube through the cervical canal.

9. The apparatus of claim 7 wherein the tube, from midway between the ends towards the distal end, is curved.

10. The apparatus of claim 7 wherein the tube is flexible.

11. The apparatus of claim 7 wherein the tip is blunt.

12. The apparatus of claim 7 wherein the tip has an aperture across the axis of the tube and at least one aperture along the axis of the tube.

13. The apparatus of claim 7 wherein the anesthetic has a viscosity greater than that of water.
